(19) United States
(12) Patent Application Publication
(10) Pub. No.: US 2026/0070706 A1
(43) Pub. Date: Mar. 12, 2026

(54) STORAGE CONTAINER WITH TORTUOUS PATH SEAL

(71) Applicant: Instant Systems, Inc., Norfolk, VA (US)

(72) Inventors: Uzair Ahmed RAJPUT, Virginia Beach, VA (US); Emilio Alejandro EDWARDS, Norfolk, VA (US)

(73) Assignee: Instant Systems, Inc., Norfolk, VA (US)

(21) Appl. No.: 19/106,801

(22) PCT Filed: Sep. 6, 2023
(86) PCT No.: PCT/US2023/032046

§ 371 Date: Feb. 26, 2025

Related U.S. Application Data
(60) Provisional application No. 63/404,234, filed on Sep. 7, 2022.

(51) Int. Cl. B65D 33/18; B65D 75/58

(52) U.S. Cl. CPC B65D 33/18; B65D 75/5805

(57) ABSTRACT

A flexible container includes a primary closure including a fold region having an adhesive material located along the opening. The fold region is configured to fold over the opening such that the adhesive material contacts a first layer or a second layer of the container to seal the opening. The container also includes a secondary closure including a tab extending past at least one of the first edge or the second edge and spaced apart from the fold region. The tab is configured to secure the fold region after the fold region is folded over the opening.

### CROSS-REFERENCE TO RELATED APPLICATIONS

This patent application claims priority to and the filing date benefit of U.S. Provisional Application Ser. No. 63/404,234, filed Sep. 7, 2022, entitled “Storage Container with Tortuous Path Seal,” which is incorporated herein by reference in its entirety.

### BACKGROUND

The embodiments described herein relate to containers for storing and transporting tissue and other biological material. More particularly, the embodiments described herein relate to devices and methods including self-sealing, flexible containers that can withstand the conditions needed for storing and transporting tissue.

Known tissue implants and/or grafts are used in a variety of procedures to repair or replace damaged tissue. Such procedures can include implanting bone or gum tissue to address dental or periodontal issues, bone grafting to repair fractures, and tendon grafting to repair damaged ligaments and/or tendons (e.g., repair of a torn anterior cruciate ligament), to name just a few. In many instances, the tissue implant is not taken from the patient's body (i.e., is not an autograft), but rather is from another source, such as from a human cadaver (i.e., an allograft) or an animal (i.e., a xenograft). Tissue is also used in a variety of research or training applications. Thus, known methods for preparing tissue implants can include multiple handling operations. Such operations can include recovering donated tissue, packaging the donated tissue for shipment to a processing center, preparation and processing of the donated tissue, and storage of the donated tissue for later shipment to a recipient.

During certain recovery operations, the tissue may be frozen, refrigerated, cryopreserved, and/or stored at ambient temperatures. The tissue may also be transported using various refrigerants, such as, for example, wet ice, dry ice, LN2 filled or LN2 vapor phase storage transport boxes. In other situations, the tissue may not be refrigerated or cooled during transportation. As such, effective tissue recovery operations require specialized containers (or container systems) that can be used in a variety of different situations. Such container systems keep the tissue therein free from contact with (or contamination by) outside sources (e.g., refrigerants, other tissues, etc.) during storage, handling, and transportation.

Unfortunately, known tissue handling procedures can result in loss of tissue due to insufficient container systems. For example, some known containers are not robust enough to prevent breaches (e.g., such known containers are susceptible to rips, tears, inadvertent opening, etc.). To limit the likelihood of breach when using flexible containers, some known container systems include one or more secondary containers to protect the inner container and contents. Such secondary containers, however, are often rigid, and therefore can be difficult to manipulate and pack, and can also inefficiently use the available space in the shippers/freezers (which can increase shipping cost). Moreover, known sealing systems and procedures that are used at the point of recovery are often insufficient to limit breach. Specifically, although known sealing methods in a centralized setting can include sophisticated heat sealers, these devices can be bulky and expensive and are therefore not suitable for use at a point of recovery. As such, in some instances it is not possible or practical to have a separate sealing device for closing and sealing the storage containers on location during the harvest or preservation of material. While there are known methods and devices for closing the containers without a separate sealing device, improvements can be made over these methods and devices. For example, the open end of storage bags can be clamped closed. This method is limited because clamps are cumbersome and take up valuable space. Additionally, clamps can fail, open on their own, break, allow leaks or complete failures. Also, clamps can be lost or forgotten as they are not an integral part of the container.

In another example, the open end of the storage bag can be twisted for closure. In some instances, the storage bag can even be twisted into a knot. However, twists can come undone. Additionally, they are not uniform in application. The lack of consistency from bag to bag can make storage difficult. There is also no guarantee of complete closure, and it can be difficult or impossible to verify or validate that the storage bag is completely closed.

In another example, the open end of the storage bag can be tied shut. Like twisting, the tied knot can come undone, and the knot would not be uniform in application. Additionally, the use of the same force to tie the storage bag would be difficult or impossible to replicate. Again, like the twisting method there is also no guarantee of complete closure, and it can be difficult or impossible to verify or validate that the storage bag is completely closed.

In another example, the open end of the storage bag can be folded, and the entire storage bag can be wrapped within another material. Sometimes this is done with a cloth. However, wrapping is not leak proof. Additionally, wrapping these bags is not uniform in practice and not validate-able. Wrapping does not keep contaminates from entering the wrapped storage bag. Wrapping also uses a secondary item, the wrap itself, which can be lost or forgotten as it is not an integral part of the bag.

In another example, the open end of the storage bag can be zipped close with an integral zipper. However, the material (e.g., tissue samples) can become lodged in the zipper mechanism. The zipper mechanism does not allow for customizing the size or fit of the storage volume as the location of the zipper dictates both. Also, the zipper requires some precision by the operator to close properly. Visual inspection can also be difficult in certain circumstances.

In another example, the open end of the storage bag can be sealed using a sealing device. However, the sealing devices require space and electricity. These devices are not generally available at the site of recovery and are not customarily brought to recover sites as they are large, heavy, and cumbersome. Ensuring a sealing device is available, working, has space and electricity are barriers to their use in this situation.

Based on the known solutions and their shortcomings, some of which are discussed above, there is a need for improved containers and methods for storing, transporting, and/or processing, units of biological material including on site closure of storage bags, and flexible containers that overcome one or more of these shortcomings.

### SUMMARY

Storage containers are described herein along with methods for storing materials including tissue and other biologicals. In some embodiments, a flexible container includes a first layer and a second layer, at least one of which is constructed from a first material. The first layer and second layer are coupled together to form the flexible container having a first end portion, a second end portion, a first edge between the first end portion and the second end portion, and a second edge between the first end portion and the second end portion. The first edge is opposite the second edge. The first layer and the second layer are coupled together along the first edge, the second edge, and the second end portion to define a storage volume between the first layer and the second layer with an opening into the storage volume defined at the first end portion. The container also includes a primary closure including a fold region having a second material located along the opening. The fold region is configured to fold over the opening such that the second material contacts the first material of one of the first layer or the second layer to seal the opening. The container also includes a secondary closure including a tab extending past at least one of the first edge or the second edge and spaced apart from the fold region. The tab is configured to secure the fold region after the fold region is folded over the opening.

In some embodiments, the fold region is formed from a portion of the first layer that extends beyond the opening. The fold region includes a fold region width. The secondary closure is spaced away from the fold region by a multiple of the fold region width. In some embodiments, the fold region extends across a full width of the first end portion. In some embodiments, the fold region includes a flap that extends past the first edge. The flap is configured to be folded onto one of the first layer or the second layer after the fold region is folded over the opening. The tab extends past the first edge and defines a connection separation region. In some embodiments, the fold region includes a perforated line defining an edge of the fold region. The fold region is configured to fold about the edge and over the opening.

In some embodiments, the first material is an extruded polymer tube. The second material is an adhesive. The fold region includes a removable adhesive liner that covers the adhesive before use. In some embodiments, the second material is more rigid than the first material allowing the second material to guide the folding of the fold region over the opening. The secondary closure includes an adhesive or a cohesive formulated to couple the tab to the fold region after the fold region is folded over the opening. In some embodiments, the first layer and the second layer define a frangible region configured for opening the flexible container after the opening is sealed by at least one of the primary closure or the secondary closure. The opening is a first opening. The frangible region includes a stress concentration riser configured to form at least one of a separation between the first layer and the second layer or a second opening through at least one of the first layer or the second layer.

In some embodiments, a method of enclosing a material in a flexible container is provided. The flexible container includes a first end portion and a second end portion. The flexible container is constructed from a first layer and a second layer coupled together to define a storage volume between the first layer and the second layer with an opening into the storage volume defined at the first end portion. The flexible container includes a primary closure and a secondary closure. The method includes separating the first layer and the second layer to form an expanded opening. The method also includes inserting the material into the storage volume via the expanded opening. The method also includes compressing the flexible container to expel air from the storage volume by applying a pressure against an outer surface of the flexible container progressively from the second end portion towards the opening. The method also includes removing a first adhesive liner from a fold region of the primary closure to expose a first adhesive. The method also includes folding the fold region along a perforation line to cover the opening. The method also includes sealing the fold region to the second layer via the first adhesive. The method also includes folding, after the sealing, the fold region along a length of the flexible container until the fold region overlaps the secondary closure. The method also includes removing a second adhesive liner from the secondary closure to expose a second adhesive. The method also includes adhering the fold region to the secondary closure via the second adhesive.

In some embodiments, folding the fold region along the perforation line includes folding the fold region from 2 to 6 times. Folding the fold region along the perforation line includes folding the fold region 4 times. The secondary closure includes a tab extending wider than a width of the flexible container. The method includes wrapping the tab of the secondary closure around the fold region when adhering the fold region to the secondary closure after folding the fold region across the secondary closure such that a portion of the secondary closure adheres to one side of the fold region and the tab adheres to a second side of the fold region.

In some embodiments, a flexible container system includes a first layer and a second layer constructed from a first material. The first layer and second layer are coupled together to form the flexible container having a first end portion, a second end portion, a first edge between the first end portion and the second end portion, and a second edge between the first end portion and the second end portion. The first edge is opposite the second edge. The first layer and the second layer are coupled together along the first edge, the second edge, and the second end portion to define a storage volume between the first layer and the second layer with an opening into the storage volume defined at the first end portion. The first layer extends past the second layer at the opening. The system also includes a primary closure including a fold region. The fold region is located along the opening and is defined by a portion of the first layer. The fold region extends past the second layer. The fold region is configured to form a first fold over the opening to seal the opening. The system also includes a secondary closure spaced apart from the fold region. The secondary closure is configured to form a secondary seal between the fold region and the second layer when the fold region is folded across the secondary closure.

In some embodiments, a kit includes a flexible container and a support stand. The flexible container has a first layer, a second layer, a first closure and a second closure. At least one of the first layer or the second layer is constructed from a first material. The first layer and second layer are coupled together to form the flexible container having a first end portion, a second end portion, a first edge between the first end portion and the second end portion, and a second edge between the first end portion and the second end portion. The first edge is opposite the second edge. The first layer and the second layer are coupled together along the first edge, the second edge, and the second end portion to define a storage volume between the first layer and the second layer with an opening into the storage volume defined at the first end portion. The first closure includes a fold region having a second material located along the opening. The fold region configured to fold over the opening such that the second material contacts the first material of one of the first layer or the second layer to seal the opening. The second closure includes a tab extending past at least one of the first edge or the second edge and spaced apart from the fold region, the tab configured to secure the fold region after the fold region is folded over the opening. The support stand is configured to maintain the flexible container in a position with the first end portion being above the second end portion. The support stand has an attachment mechanism configured to be coupled to at least one of the first closure or the second closure to removably couple the flexible container to the support stand.

### BRIEF DESCRIPTION OF THE DRAWINGS

FIG. 1 is a schematic illustration of a storage container according to an embodiment.

FIG. 2 is a schematic illustration of the storage container according to FIG. 1 with a material being moved into the storage container.

FIG. 3 is a schematic illustration of the storage container according to FIG. 1 containing the material of FIG. 2 with air being expelled.

FIG. 4 is a front view of the storage container according to FIG. 1 containing the material of FIG. 2.

FIG. 5 is an enlarged view of a portion of the storage container according to FIG. 4 showing the first and second closure.

FIG. 6 is a detailed schematic illustration of the storage container according to FIG. 4 showing the first closure folded over a first time.

FIG. 7 is a detailed schematic illustration of the storage container according to FIG. 4 showing the first closure folded over three times.

FIG. 8 is a detailed schematic illustration of the storage container according to FIG. 4 showing the second closure folded around side edges of the storage container.

FIG. 9A is a schematic illustration of a storage container according to an embodiment.

FIG. 9B is a schematic illustration of a storage container according to an embodiment.

FIG. 10 is a schematic illustration of a storage container system according to an embodiment.

FIG. 11 is a schematic illustration of a storage container according to an embodiment.

FIG. 12 is a flow chart showing a method of storing a material according to an embodiment.

### DETAILED DESCRIPTION

The embodiments described herein related to a storage container and methods of use that can advantageously be used in a wide variety of material storage, transportation, processing and/or implantation operations. In particular, the storage containers described herein can allow for material (e.g., a tissue specimen, biologic material, therapeutics etc.) to be loaded. The storage containers are then sealed at the point of loading.

In some embodiments, an apparatus includes a container that is robust for storing tissue and does not require secondary containment. The container includes a self-sealing portion that allows the container to be sealed at a variety of locations (e.g., a surgical suite or the like) without specialized equipment. The self-sealing portion can include any suitable mechanism that provides a repeatable seal that limits penetration of microbes therethrough.

In accordance with various embodiments, a storage container can include a closure mechanism. The closure mechanism is structured such that it is easy to determine that it is properly closed. In some examples, the closure mechanism can include a fold region configured to fold over the opening. The fold region can also be configured such that similar folds are repeatable between different storage containers. Various indicia, guides, lines of weakness, or other suitable mechanisms can be used to provide repeatability of folds. Additionally or alternatively, the storage container can include a self-sealing mechanism. The self-sealing mechanism can include one or more seals suitable to maintain the closure. For example, the self-sealing mechanism can include an end closure. The self-sealing mechanism can also include an intermediate closure that is applied to the storage container without the need for a sealer (i.e., an external sealing apparatus, such as a heat sealer). The combination of folding mechanism and the self-sealing mechanism provides a (1) tortious path created by the folding of one or more layers of the storage container and (2) one or more self-sealing seals that secure the tortious path. Such a structure can also be configured to produce visual verification that that tissue is securely sealed within the container (e.g., via a color change or the like). The structure can provide customization of the storage volume that allows for additional security of specimen and limits the space provided to the material within the storage volume. The structure limits the likelihood that the specimens therein will get lost, contaminated, destroyed, or interfered with during transit of the storage container before the material is added or transit of the storage container system container the material after sealing. The structure including multiple self-sealing seals secures the tortious path thereby keeping pathogens out and biologicals within the container.

As used herein, the term “about” when used in connection with a referenced numeric indication means the referenced numeric indication plus or minus up to 10% of that referenced numeric indication. For example, the language “about 50” covers the range of 45 to 55. Similarly, the language “about 5” covers the range of 4.5 to 5.5.

As used herein, the term “biologic material” refers to any material that is produced or derived from a living (or recently living) organism. Biologic materials can include, for example, tissue specimens, tissue grafts, cells, blood, or other bodily fluids. Biologic materials can also include plants, plant products, micoorganisms, genetically modified organisms (including cells and cell lines). Biologic materials can also include DNA or RNA (including plasmids, oligonucleotides, cDNA) or viral vectors. Biologic materials can also include material that is produced by a living (or recently living) organism, such as small or large molecule pharmaceuticals.

As used herein, the term “tissue specimen” or “tissue graft” refers to any material that can be used in a tissue repair procedure or other procedures which use tissue grafts (e.g., birth tissue used as patch for healing then removed). Thus, a tissue specimen or a tissue graft can include any of a skin graft, bone tissue, fiber tissue (e.g., tendon tissue, ligament tissue, or the like), ocular tissue (e.g., corneal implants), birth tissue (e.g., amnion graft), cardiovascular tissue (e.g., heart valve), tendons or the like including artificially produced tissue. A tissue specimen or a tissue graft can include a portion of tissue harvested from a donor or a structure component that includes both tissue and non-tissue material (e.g., a synthetic matrix that includes tissue therein). For example, a tissue specimen or a tissue graft can include bone tissue that also includes bone cement or other non-tissue components. As another example, a tissue specimen or tissue graft can include bone chips including cortical bone chips, cancellous bone chips, and corticocancellous bone chips, and/or bone chips with viable bone lineage committed cells.

As used herein, the term “stiffness” relates to an object's resistance to deflection, deformation, and/or displacement produced by an applied force, and is generally understood to be the opposite of the object's “flexibility.” For example, a layer or structure of a container with greater stiffness is more resistant to deflection, deformation and/or displacement when exposed to a force than is a layer or structure of the container having a lower stiffness. Similarly stated, a container (or layer) having a higher stiffness can be characterized as being more rigid than a container (or layer) having a lower stiffness. Stiffness can be characterized in terms of the amount of force applied to the object and the resulting distance through which a first portion of the object deflects, deforms, and/or displaces with respect to a second portion of the object. When characterizing the stiffness of an object, the deflected distance may be measured as the deflection of the portion of the object different than the portion of the object to which the force is directly applied. Said another way, in some objects, the point of deflection is distinct from the point where the force is applied.

Stiffness (and therefore, flexibility) is an extensive property of the object being described, and thus is dependent upon the material from which the object is formed as well as certain physical characteristics of the object (e.g., cross-sectional shape, thickness, boundary conditions, etc.). For example, the stiffness of an object can be increased or decreased by selectively including in the object a material having a desired modulus of elasticity, flexural modulus and/or hardness. The modulus of elasticity is an intensive property of (i.e., is intrinsic to) the constituent material and describes an object's tendency to elastically (i.e., non-permanently) deform in response to an applied force. A material having a high modulus of elasticity will not deflect as much as a material having a low modulus of elasticity in the presence of an equally applied stress. Thus, the stiffness of the object can be decreased, for example, by introducing into the object and/or constructing the object of a material having a relatively low modulus of elasticity. Similarly, the flexural modulus is used to describe the ratio of an applied stress on an object in flexure to the corresponding strain in the outermost portions of the object. The flexural modulus, rather than the modulus of elasticity, is often used to characterize certain materials, for example plastics, that do not have material properties that are substantially linear over a range of conditions. An object with a first flexural modulus is more elastic and has a lower strain on the outermost portions of the object than an object with a second flexural modulus greater than the first flexural modulus. Thus, the stiffness of an object can be reduced by including in the object a material having a relatively low flexural modulus.

Moreover, the stiffness (and therefore flexibility) of an object constructed from a polymer can be influenced, for example, by the chemical constituents and/or arrangement of the monomers within the polymer. For example, the stiffness of an object can be reduced by decreasing a chain length and/or the number of branches within the polymer. The stiffness of an object can also be reduced by including plasticizers within the polymer, which produces gaps between the polymer chains.

The stiffness of an object can also be increased or decreased by changing a physical characteristic of the object, such as the shape or cross-sectional area of the object. For example, an object having a length and a cross-sectional area may have a greater stiffness than an object having an identical length but a smaller cross-sectional area. As another example, the stiffness of an object can be reduced by including one or more stress concentration risers (or discontinuous boundaries) that cause deformation to occur under a lower stress and/or at a particular location of the object. Thus, the stiffness of the object can be decreased by decreasing and/or changing the shape of the object.

As used in this specification, specific words chosen to describe one or more embodiments and optional elements, or features are not intended to limit the invention. For example, spatially relative terms—such as “beneath.” “below”, “lower”, “above”, “upper”, “proximal”, “distal”, and the like—may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations.

Similarly, geometric terms, such as “parallel,” “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

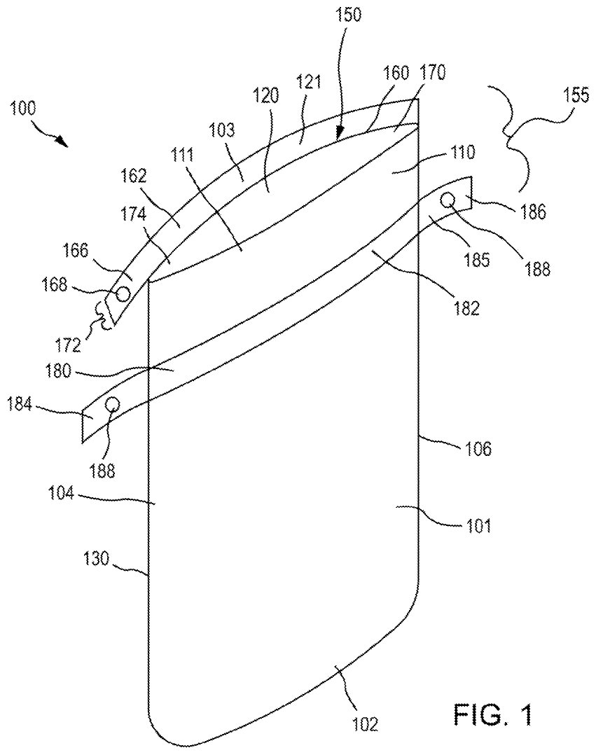

An embodiment of a storage container 100 is shown in FIG. 1. The storage containers discussed herein including any suitable package, pouch, bag, or container assemblies includes internal volumes configured to suitably store one or more of the materials 50. The storage containers can include in some embodiments a flexible material forming the storage container 100 as a flexible container. The storage container 100 (and any of the container assemblies described herein) includes a storage volume 101 that can be used to store the material 50 and/or to perform any of the methods described herein and/or the methods of preparing the material 50 for use in a procedure. As described herein, the storage container 100 provides a container that can be used for cryogenically freezing, storage, transport, processing, defrosting and/or rehydration of the material 50. The storage container 100 also includes a closure mechanism 155 suitable to close any opening into storage volume 101. In some examples, the closure mechanism 155 is structured such that it is easy to verify that the container is sealed.

As used herein the materials 50 can include compounded and non-compounded pharmaceuticals, a tissue or tissues, cellular material, biological material (including but not limited to biological material G, as described herein), and/or related media (herein referred to together as stored product). In some embodiments, the container assemblies described herein can be used to store packages containing tissue, cellular material, biological material, or related media. In some embodiments, the stored product within internal volumes or within packages within internal volumes can include biologic materials, including but not limited to, human and animal tissues, human and animal cells or cellular materials, plant materials (tissue and cellular materials), organs, organoids, biologically sourced materials (e.g., printed tissues, cells, organs, or organoids), bacteria, viruses, viral vectors, fungi, medical devices, combination devices, material for homologous or non-homologous use, and/or materials for autologous or allogenic use. In some embodiments, the stored product can include cellular material, including but is not limited to, lineage committed, and non-lineage committed cells (e.g., bone lineage committed cells, osteoblasts, osteocytes, etc.), differentiated cells or non-differentiated cells (e.g., muscle cells, endothelial cells, etc.), and/or genetically modified or non-genetically modified materials. Examples of human and animal tissues include, but is not limited to, birth tissues (e.g., amnion, cord, cord blood, chorion, placenta, etc.), bones and/or products made from bones (e.g., machined allografts, ground particles, etc.), bone sources (e.g., tibia, fibula, humerus, cranial flaps, radius, ulna, pelvic bones, and joints, etc.), brain tissue, cartilages (from all sources in bodies generally from knee joints, shoulders, etc.), fascia lata, heart valves, arteries, veins, nerves, organs (e.g., lungs, hearts, liver, kidneys, etc.), reproduction tissue (e.g., semen and eggs), ribs, soft tissues (e.g., all tendons, Achilles, patellar, etc.), skin, and/or tumors. Examples of the human or animal cellular materials include, but is not limited to, B-cells, blood cells and blood derived cells, bone cells, CAR-T cells, egg cells, engineered T-Cells, fat cells, muscle, cells, natural killer cells, nerve cells, sperm cells, stem cells (modified and un-modified, differentiated, and non-differentiated), T-cells, tumor infiltrating lymphocytes (TIL), viral vectors, viruses and bacteria. The human or animal cellular material can be modified or non-modified (such as genetically modified). Examples of the plant materials include, but is not limited to, cellulose, hemicellulose, pectin, fruit, fungi, leaves, mitochondria, plant organelles, pollen, roots, seeds, shoots, and/or stems. In some embodiments, the stored product can include related media, including but not limited to, culture media, saline solution, cryoprotectant, preservation solution, etc. It will be appreciated that any suitable stored product can be stored within any of the container assemblies described herein.

In accordance with some embodiments and illustrated in FIG. 1, the storage container 100 also includes a first layer 110 and a second layer 120 that are coupled together. The storage container 100 also includes a side edge 104 and a side edge 106. In some embodiments, these side edges 104 and 106 are opposite each other. The storage container 100 also includes a first end portion 103 and a second end portion 102. The first layer 110 and the second layer 120 are coupled together along the side edge 104, the side edge 106, and the second end portion 102 to define a storage volume 101 between the first layer 110 and the second layer 120 with an opening 150 into the storage volume 101. The opening 150 can be located at the first end portion 103.

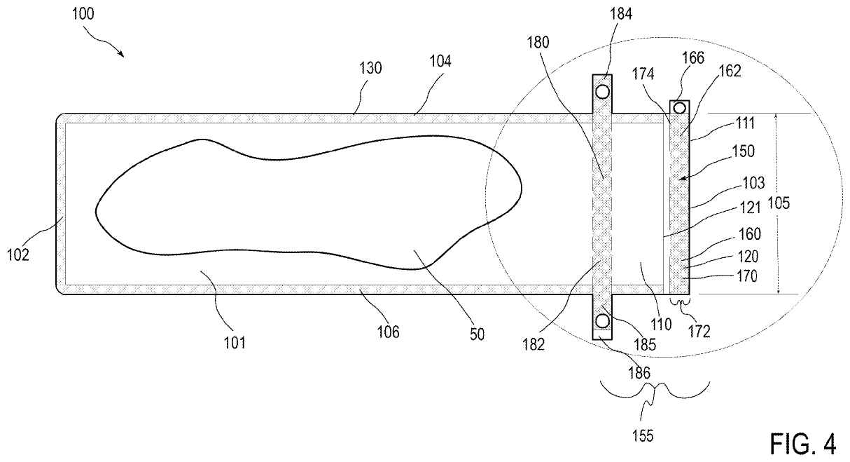

In accordance with some embodiments, first layer 110 and the second layer 120 are coupled together via any suitable connection structure. For example, they can be coupled together by one or more seals 130 (see, e.g., FIG. 4). The one or more seals 130 can form the side edges 104, 106 of the storage container 100. The one or more seals 130 can be located near the side edges 104, 106. While sealed edges can define the storage container 100, it is also appreciated that in some embodiments, the edges are coupled together by any suitable mechanism or can be a monolithically formed. For example, the first layer and the second layer can be contiguous such as is the case with a tubular material that is flattened forming two longitudinal connections between the layers on the flattened longitudinal edges of the tubular material (e.g., layflat tubular film). In such embodiments, the tubular film can be flattened out with the edges of the flattened tube forming the side edges 104, 106.

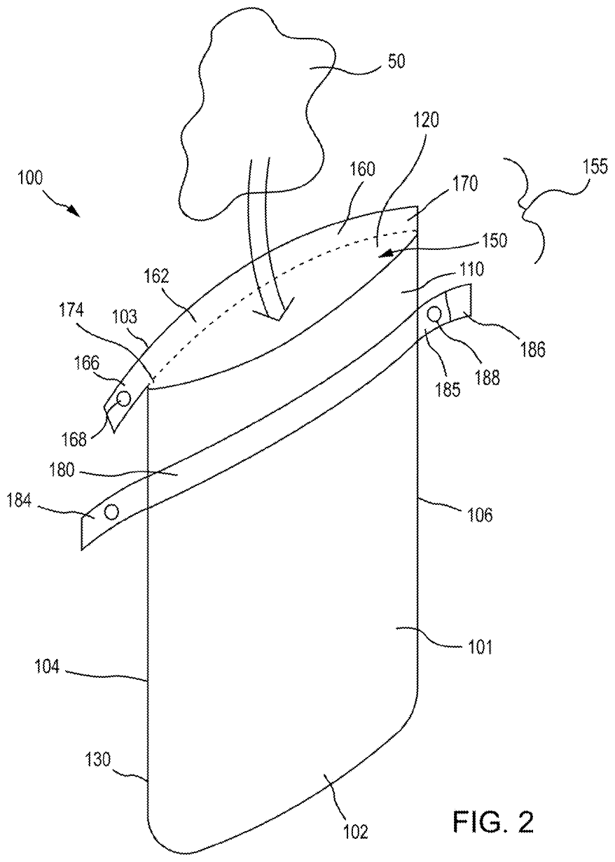

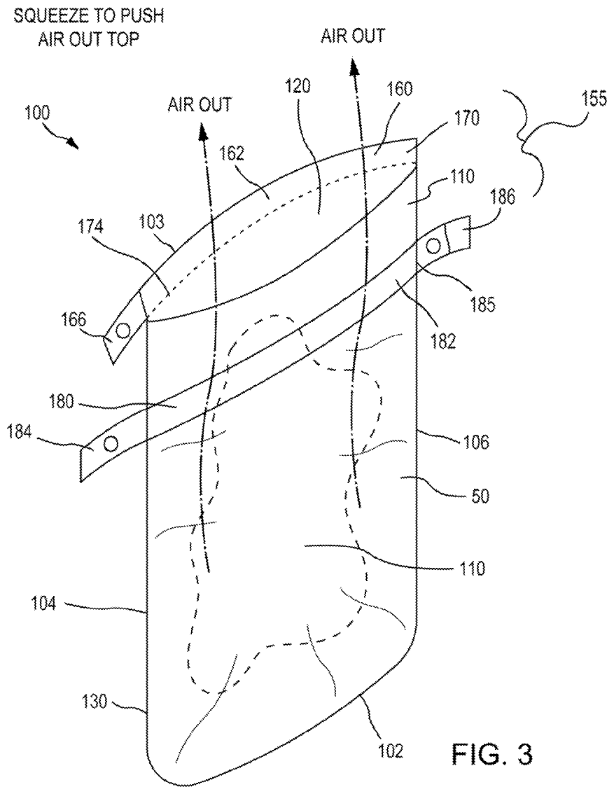

As shown in FIG. 1, the storage container 100 includes one or more openings 150. The opening 150 can be suitable for material egresses or ingress. The opening 150 can allow the storage container 100 to be placed in an open configuration. In the open configuration one or more edges at the first end portion (e.g., edge 111) of the first layer 110 are spaced apart from one or more edges at the first end portion (e.g., edge 121) of the second layer 120 to define an opening 150 into the storage volume 101 of each of the storage container 100. The opening 150 can be of any suitable size to facilitate loading of the material 50 (including e.g., tissue, biological material and/or treatment therapeutics) such that the volume 101 can receive the material 50. In some embodiments, the opening 150 can extend across a portion of the length of an end or a side of the storage container 100. In some embodiments, the opening 150 can extend across substantially all of the end (e.g., the first end portion 103) or side of the storage container 100. In the example shown in FIG. 1, the opening 150 can extend across the edge 111 of the storage container 100 defined by the separation between layers 110 and 120. In some embodiments, the opening 150 is sufficiently large to allow for input of a larger material (e.g., a tissue graft) into the storage volume 101. Although it is appreciated that other materials as discussed herein can additionally or alternatively be introduced through opening 150. As shown in FIG. 2, the material 50 is introduced into the storage volume 101 through the opening 150. After the material 50 is introduced into storage volume 101 (see FIG. 3), the opening 150 can be closed by the closure mechanism 155 as shown in FIGS. 4-8. Sealing the opening 150 by the closure mechanism 155 encloses storage volume 101 isolating the material 50 therein.

As illustrated in FIGS. 4-8 and in accordance with some embodiments, the closure mechanism 155 can include an end closure 160 suitable to close opening 150. Additionally or alternatively the closure mechanism 155 can include an include an intermediate closure 180. In embodiments having both an end closure 160 and an intermediate closure 180, the end closure 160 can be a primary closure suitable to provide at least some material retention and/or security. The intermediate closure 180 (which can function as a secondary closure) can be applied after the end closure 160 and provides increased retention and/or security.

In accordance with some embodiments, the end closure 160 closes off the first portion 103 of the storage container 100. The end closure 160 is located proximal to the end portion 103 and is configured to close the separation of edges 111 and 121. Similarly stated, the end closure 160 is configured to cover and/or seal the opening 150. In some embodiments, the end closure 160 includes a fold region 170 that allows for folding one or both layers (e.g., 110, 120) over thereby closing the opening 150.

In accordance with some embodiments, the fold region 170 is defined within the first end portion 103 and is configured to allow for consistent folding of the one or more layers. The folds can be consistent between multiple folds on a single storage container 100. The folds can be consistent between different containers as well, allowing uniformity in the way the different containers are sealed. The fold region extends across a full width of the first end portion. The fold region 170 can include a fold guide 174 to indicate to the user loading the material 50 into the storage container 100 where to fold the end closure 160. In some examples, the fold guide 174 can include indicia on one or both layers 110, 120. When folded along the indicia, the fold crease forms a closure across at least a portion of the opening 150. In some embodiments, the fold guide 174 can include a line of weakness making it easier to create a crease and begin the folding process. The line of weakness can include abrasions in the material, a thin line of distortion formed in one or both layers, a cut extending partially through one or both layers, perforations along a portion of the width of the material, stress risers, or other suitable feature to mark and allow easier initiation of the fold. In some examples, the fold guide 174 can include a second material that is more rigid than the first and/or second layer (110, 120). The second material can define the fold region. A user can utilize the rigidity of the second material to assist in folding the first layer or the second layer around its edges. Specifically, the rigid second material can have a defined width that functions to guide repeatable folding.

In accordance with some embodiments, the first layer and the second layer 110, 120 can extend different lengths at the first end portion 103. For example, one layer (e.g., layer 120) can extend past the other layer (e.g., layer 110) at the opening (See FIG. 1). In some examples, this additional length of one layer (e.g., 120) extending past the other layer (e.g., 110) can define the fold region 170. In some examples, the fold region 170 can include just a portion of the length of one layer (e.g., 120) extending past the other layer (e.g., 120). The extended layer can be folded back on the first layer forming the end closure 160.

In some embodiments, the end closure 160 includes a connector 162. The connector can include a chemical or mechanical connection suitable to attach one layer of the container 100 to the other layer or to the same layer. In some embodiments, the connector 162 is a second material such as an adhesive or cohesive. For example, the end closure 160 can include an adhesive that extends over part of or all of the fold region 170 such that when fold region 170 is folded back, the adhesive secures the fold in place limiting its ability to unfold. As the fold region 170 can be located along the opening 150, the fold region 170 folds over the opening 150 such that the adhesive contacts the outer layer (e.g., layer 110 as shown in FIG. 6) thereby sealing the folded portion to the layer and closing the opening 150.

The fold region is characterized by a fold region width 172. The fold region width 172 defines the overlap of the first fold. In accordance with some embodiments, the fold region 170 can be folded over more than once. Each successive fold would be about the width of the fold region width 172. Each successive fold can also add an additional crease in one or both layers. The additional crease further limits the ability of the material to egress from the storage volume 101. Multiple folds create a tortious path that the material 50 (or any solutions or preservatives) would have to traverse to egress from the storage volume 101 thereby further securing the closure. Similarly, the tortious paths defined by the multiple folds limits or prevents the ingress of environmental contaminants (e.g., microbes). Additionally, the size of the storage volume 101 can be controlled by adapting the number of folds to the desired size. The smaller the desired size, the more folds the user can use. The larger the designed size, the fewer the folds the user would use. In one example the user folds the fold region more than once. In another example the user folds the fold region from 2 to 6 times. In another example as shown in the transition fold between FIG. 7 and FIG. 8, the user folds the fold region 4 times.

In some embodiments the end closure 160 can include a tab 166 that extends past the first edge 104. In some examples, the tab 166 is configured to be folded onto one of the first layer or the second layer after the fold region is folded over the opening 150. This can further secure the fold after folding and also close any opening along the side edge 104. While only shown as extending from one side, it is appreciated that tabs can extend past either or both of edges 104 and 106. Additionally or alternatively, the tab 166 is a peelable extension of the peel and stick adhesive that forms a part of the closure mechanism 160. In such an example, in order to apply the closure mechanism 160 adhesive, the user would remove (e.g., peel back) a covering over a peel and stick adhesive. This exposes the adhesive that can then be attached to one of layers 110 or 120.

In accordance with some embodiments, the intermediate closure 180 can operate in conjunction with of the storage container 100 as a secondary closure or in other embodiments the intermediate closure 180 can independently seal the material 50 in storage volume 101. The intermediate closure 180 can include a chemical or mechanical connection suitable to attach one layer of the container 100 to the other layer or to the same layer. In some embodiments, the chemical connection is an adhesive or cohesive. For example, the intermediate closure 180 can include an adhesive that extends across one of the layers (e.g., 110 or 120) such that when the container is folded back on itself (e.g., when the end closure 160 folds the container), the adhesive secures the fold in place limiting its ability to unfold. In embodiments in which the intermediate closure 180 operates in conjunction with end closure 160, the fold region 170 contacts along the outer layer (e.g., layer 110 or 120) of the folded portion thereby sealing the folded portion to the layer and further sealing and securing the material 50 within storage volume 101.

In some embodiments, the intermediate closure 180 is spaced away from the fold region by a multiple of the fold region width 172. For example, the intermediate closure 180 is located from one to seven widths 172 (i.e. fold-region widths) from the edge 111. For example, the intermediate closure 180 can be located between three to five widths from the edge 111. In one example, the intermediate closure 180 is located two widths from the edge 111. In one example, the intermediate closure 180 is located three widths from the edge 111. In the example shown in FIG. 4, the intermediate closure 180 is located as the fourth fold region width 172 from the edge 111.

In some embodiments, the intermediate closure 180 can include one or more tabs (e.g. tabs 184 and/or 185) that extends past one or more of the edges (e.g., edges 104 and/or 106). In some examples, the tabs (e.g. tabs 184 and/or 185) are configured to be folded onto one of the first layer or the second layer after the first layer or the second layer are folded over the intermediate closure 180. This can further secure the fold after folding. Additionally or alternatively, one or both of the tabs (e.g. tabs 184 and/or 185) can include a peelable extension of the peel and stick adhesive that forms a part of the intermediate closure 180. In such an example, in order to apply the intermediate closure 180 adhesive, the user would remove (e.g., peel back) a covering over a peel and stick adhesive. This exposes the adhesive that can then be attached to one of the layers 110 or 120 that have been folded across intermediate closure 180. In some embodiments, the adhesive can be a color changing adhesive suitable to provide a visual indication that seal is properly applied. For example, the adhesive changes from a first color such as red to a second color such as green in response to sufficient closure pressure on the adhesive or upon full curing of the adhesive.

In some embodiments, one or more of the tabs (e.g., tabs 166, 184, or 185) can include other structural components. For example, the tabs (e.g., tabs 166, 184, or 185) can include apertures (e.g., apertures 168 and/or 188) suitable to work with hangers on medical stands.

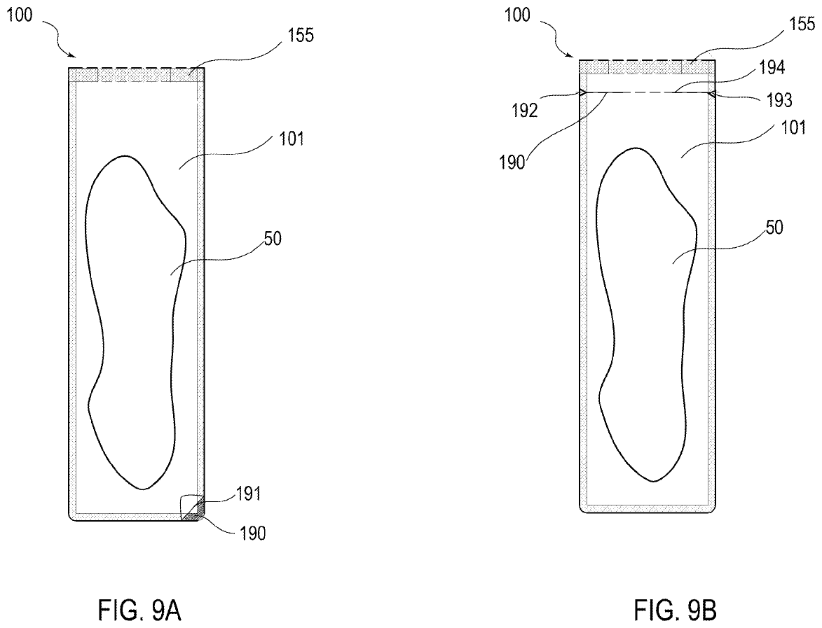

In some embodiments, the storage container 100 includes a frangible region 190 suitable for opening the storage container 100 after it has been sealed closed. FIGS. 9A-B each show an example of a sealed storage container 100 in accordance with one or more of the embodiments discussed above along with a frangible region 190 suitable to open the container 100. The frangible region 190 is a region that facilitates the opening of the storage container 100 for access to storage volume 101 in a manner other than via the opening 150. In some embodiments, the connection between the first layer 110 and the second layer 120 can include a peelable connection such that the frangible region includes areas in which the first layer and the second layer can be peeled apart after connection. For example, FIG. 9A illustrates a frangible region 190 including a peelable separation region 191. The peelable separation region 191 allows for the separation of the first layer 110 and the second layer 120. The initiation of this peel allows for the two layers to be at least partially separated and in some embodiments entirely separated.

In other examples, the frangible region 190 can be a stress-concentration riser 192, 193 as illustrated in FIG. 9B. The stress-concentration riser can include any suitable feature to initiate a tear across the volume. As illustrated in the embodiment of FIG. 9B, this can include tick perforations (or cutouts) at the edges of the volume 101 with sharp points suitable to initiate a tear into the storage volume 101 to access the material therein. In some embodiments, the tick perforations forming the frangible region 190 is a V-shaped perforation.

In other examples, the frangible region 190 can be lines of weakness 194 that extend partially or fully across the storage container 100 as illustrated in FIG. 9B. The line of weakness 194 can include abrasions in the material, a thin line of distortion formed in one or both layers, a cut extending partially through one or both layers, perforations along a portion of the width of the material, or similar structures in the storage container 100 that facilitates tearing the material to access the storage volume 101 and material therein after sealing.

As illustrated in the example of FIG. 9B, different frangible region 190 types can be combined. For example, as illustrated in FIG. 9B a partial cut can extend across the layers 110 and 120 and each end of that cut can include stress concentration riser 192, 193. Each of these frangible features can be used individually as well.

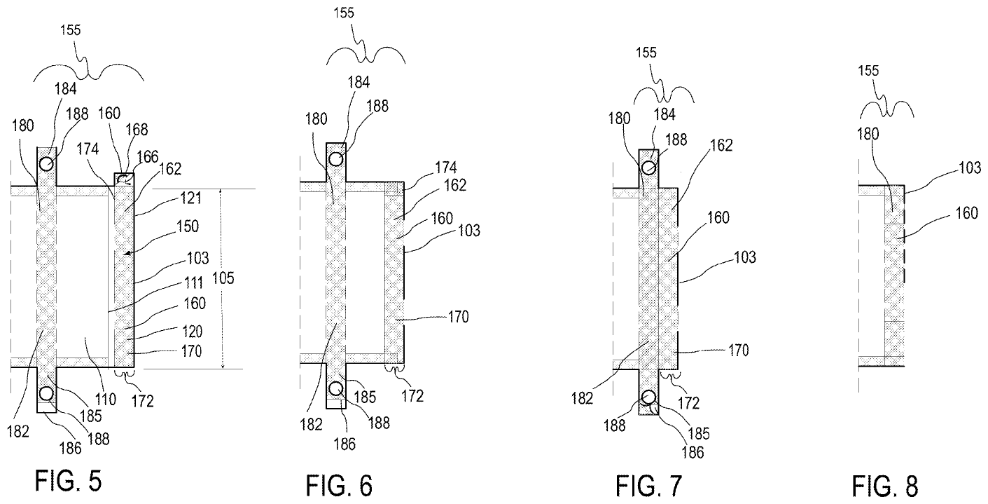

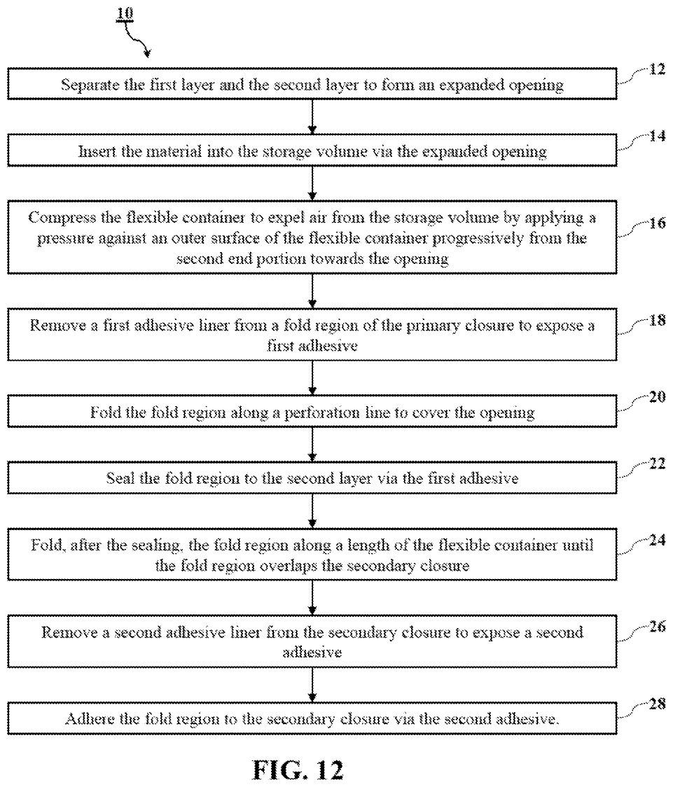

FIG. 12 is a flow chart showing a method 10 of storing a material 50 in a flexible container 100 according to one embodiment. The flexible container 100 includes a first end portion and a second end portion. The flexible container 100 is constructed from a first layer and a second layer coupled together to define a storage volume between the first layer and the second layer with an opening into the storage volume defined at the first end portion. The flexible container also includes a primary closure 160 and a secondary closure 180 (See also e.g., FIGS. 1-8). The method 10 includes separating (at 12) the first layer and the second layer to form an expanded opening (See also e.g., FIGS. 1-2). The method also includes inserting (at 14) the material into the storage volume via the expanded opening (See also e.g., FIGS. 2-3). The method also includes compressing (at 16) the flexible container to expel air from the storage volume by applying a pressure against an outer surface of the flexible container progressively from the second end portion towards the opening (see also e.g., FIG. 3). This allows the material to occupy the volume with little or no air (see also e.g., FIG. 4). The method also includes removing (at 18) a first adhesive liner from a fold region of the primary closure to expose a first adhesive (See also e.g., FIG. 5). The method also includes folding (at 20) the fold region along a perforation line to cover the opening. The method also includes sealing (at 22) the fold region to the second layer via the first adhesive (See also e.g., FIG. 6). The method also includes folding again (at 24), after the sealing, the fold region along a length of the flexible container until the fold region overlaps the secondary closure (See also e.g., FIGS. 7-8). The method also includes removing (at 26) a second adhesive liner from the secondary closure to expose a second adhesive (See also e.g., FIG. 7). The method also includes adhering (at 28) the fold region to the secondary closure via the second adhesive (See also e.g., FIG. 8).

In accordance with some embodiments, folding the fold region along the perforation line can include folding the fold region from 2 to 6 times. Preferably, folding the fold region along the perforation line includes folding the fold region 4 times. The secondary closure can include a tab extending wider than a width of the flexible container. The method can further include wrapping the tab of the secondary closure around the fold region when adhering the fold region to the secondary closure after folding the fold region across the secondary closure such that a portion of the secondary closure adheres to one side of the fold region and the tab adheres to a second side of the fold region.

In some embodiments, once the material is in the storage volume some or most of the air is removed from the container. This can be done by expelling the air from the opening 150 as shown in FIG. 3. In some embodiments, the storage container can include a second opening such as a one-way valve or a closeable port that can be used to remove the air after the storage container is sealed.

In some embodiments, the storage container 100 can include an additional opening (not shown). For example, the first opening can include a port. In other examples the first opening can include a capped opening, unattached layers, a resealable opening, or any other suitable passage for receiving or expelling the material 50, In some embodiments the additional opening can be proximal to the end portion opposite the opening 150. The additional opening can be coupled to the storage container 100 and be configured to allow fluid communication between the outside of the storage container 100 and the storage volume 101. In this manner, additional material can be inserted or additionally or alternatively material already sealed within the storage container 100 can be treated (e.g., with a preservation fluid) after being sealed within the storage container 100. Additionally or alternatively, the additional opening can also be coupled to a vacuum source to evacuate the storage volume. Additionally or alternatively, when in use (e.g., during a surgical procedure), the additional opening can allow for cryogenically defrosting the material 50. Additionally or alternatively, when in use (e.g., during a surgical procedure), the port can allow for inflow of fluid for rehydration or other purposes. Additionally or alternatively, when in use, the additional opening can allow for removal of fluid. The additional opening can be any suitable port that selectively provides fluid communication to the storage volume 101. For example, the additional opening can include a tube, a valve, and/or a cap. In some embodiments, the additional opening can be a needle-free port. In some embodiments, the additional opening can be a swabable connector. Similarly stated in some embodiments, the additional opening can have external surfaces and can be devoid of recesses or crevices such that the additional opening can be easily wiped or “swabbed” to maintain sterility during use. In some embodiments, the port can include any of the barbed, swabable valves produced by the Halkey-Roberts Corporation, such as the 2455 series of swabable valves. In other embodiments, the additional opening (and any of the ports described herein) need not be either a swabable connector or a needle-free port. Any suitable port can be employed. In some embodiments, the additional opening can include a male or female luer fitting. In some embodiments, the additional opening can include a threaded connection for coupling with a syringe or a tubing set or a cap. In some embodiments, the additional opening can be sealed at the end for sterile docking or cutting. In other embodiments, the additional can be coupled at any location and to any portion of the storage container 100. For example, in some embodiments, the additional opening need not be coupled to the second end portion of the storage container 100, the port can be on the surface or another edge portion of the storage container 100. The additional opening can be offset from a center line of the storage container 100. For example, in some embodiments, the port can be located at a corner of the container. In some embodiments, the port (and any of the ports described herein) can be coupled around a central portion of the container. In some embodiments, the port can include a one-way valve such that it can allow material in or out but not both. The port can allow air out such that the storage volume 101 only contains the material 50. For example, as part of the filling process the air can be burped out of the port after the first seal is closed.

In some embodiments, the storage container 100 and the storage volume 101 therein can be different shapes. For example, as shown in FIGS. 1-9, storage container 100 can be generally rectangular. In another example, storage container 100 can be tapered from one end portion 103 the maximum width to a narrower width on the opposite end portion 102. In some embodiments, the narrower width can be a point. In some embodiments, the narrower width can be a truncated end suitable to dispense the material as a defined flow. The tapered portion with its narrow end can be opened thereby allowing the material 50 in volume 101 to be dispensed in a controlled manner. Without a port attached to storage container 100, port leaking can be avoided producing robust long-term storage. The storage container 100 can be dispensed by opening the tip. In response to the tip being opened the material 50 can be released or dispensed in a controlled manner during dispensing of the material 50 in quality control operations. The tip can be opened by tearing, pealing, cutting, or any other suitably manner to forming an opening that allows for controlled dispensing of the material 50. In some embodiments, the edges 104 and 106 can be asymmetrical. In some embodiments, edges 104 and 106 can be non-linear. In some embodiments, the storage container can be round. The storage container can be any suitable shape that allows for material storage and suitable closure and sealing without an external sealing mechanism.

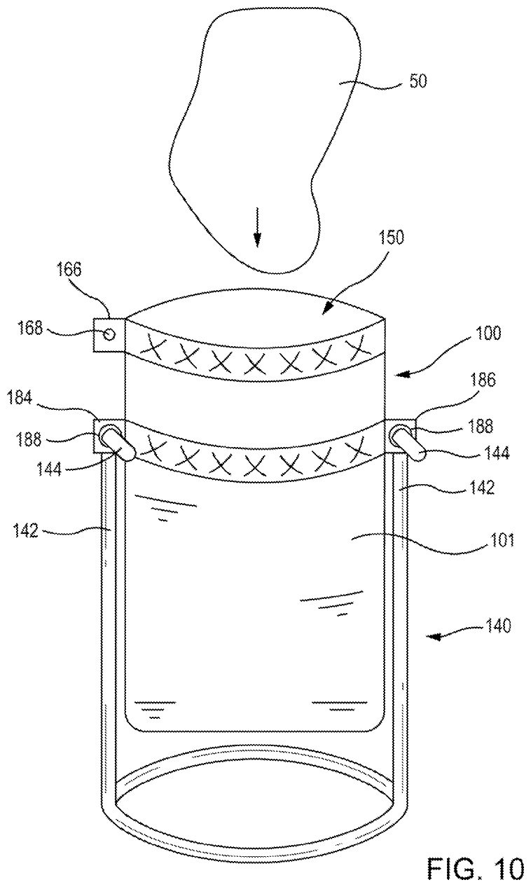

FIG. 10 is a schematic illustration of a storage container system. In accordance with some embodiments, a storage container system can include one or more of the storage container 100, the material 50, and a support stand 140. The support stand 140 can include one or more vertical structures 142 to hold the storage container 100 upright. The support stand 140 can include one or more stand attachment mechanisms 144 to engage with support features on the storage container 100. In one example, the tabs 184 and/or 185 can operate as the attachment mechanism on the storage container or the tabs 184 and/or 185 can include additional features such as apertures for hanging over the stand attachment mechanism 144. Two of the stand attachment mechanisms 144 can be sufficiently close together to force the layers 110 and 120 apart so that the bag is easier to fill. For example, the stand attachment mechanism 144 can be narrower in width than the width between the container attachment mechanisms e.g., apertures as shown in FIG. 10. In this way the opening 150 of the storage container 100 is held open.

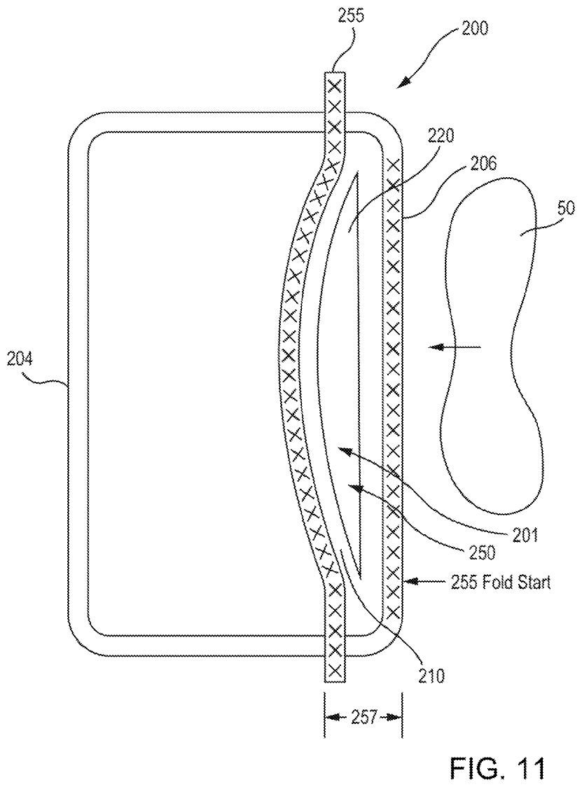

FIG. 11 is a schematic illustration of a storage container 200 showing an alternate opening 250. The storage container 200 includes one or more openings 250 and a closure mechanism 255. The opening 250 can be suitable for material egresses or ingress. The opening 250 can allow the storage container 200 to be placed in an open configuration. Instead of the opening being on the end portion as discussed above or a side of the storage container, the opening can be a slit in surface of one of the layers (e.g., surface 210). For example, the opening 250 can be a slit near one of the edges but through the surface as shown in FIG. 11. In the open configuration, a portion of the first layer 210 is spaced apart from the second layer 220 to define an opening 250 into the storage volume 201. This could be near the side edge 206. The closure mechanism 255 can be similar to those discussed above. Here, the opening is in a different location and orientation but the closure mechanism 255 would still be parallel to the opening. This configuration can allow a bigger opening size in examples in which the side dimension is larger than the width of the container. The opening would also not have to extend the entire width or length of the storage container. The opening 250 could be any location within the closing region 257 that the folds encompass. For example, if the folds encompass, 5.1 cm (2 inches) into the storage volume 201, the opening 250 could be at 4.8 cm (1.9 inches)—and it would be sealed.

The first layer 110 and/or the second layer 120 can be constructed of any suitable material. The first layer can have a first stiffness and the second layer can have a second stiffness. In some embodiments, the stiffnesses of the first layer and the second layer are the same. In some embodiments the stiffnesses are different. In some embodiments the second stiffness is greater than the first stiffness. In some embodiments, the first stiffness is greater than the second stiffness. In some embodiments, the layers can be constructed from the same material. In some embodiments, the layers can be constructed from a different material. In some embodiments, the layers can have different stiffness. In some embodiments, the first layer can be a thin, peelable film. The first layer can have any suitable thickness to provide the desired strength, flexibility, and sealing characteristics. For example, in some embodiments, the first layer 110 can be between about 10 microns (0.010 mm) and about 2000 microns (2.0 mm). In some embodiments, the first layer 110 can be between about 50 microns (0.050 mm) and about 200 microns (0.200 mm). In some embodiments, the first layer can be between about 50 microns (0.050 mm) and about 1000 microns (0.100 mm). The second layer can have any suitable thickness to provide the desired strength, flexibility, and sealing characteristics. For example, in some embodiments, the first layer can be between about 10 microns (0.010 mm) and about 2000 microns (2.0 mm). In some embodiments, the second layer 1120 can be between about 50 microns (0.050 mm) and about 200 microns (0.200 mm). In other embodiments, the second layer 120 can be between about 50 microns (0.050 mm) and about 1000 microns (0.100 mm).

In some embodiments, the layers can be produced out of any one or more of the following materials: polyethylene (PE), low density polyethylene (LDPE), composites of LDPE, linear low-density polyethylene (LLDPE), high density poly ethylene (HDPE), polychlorotrifluoroethylene (PCTFE), ethy lene tetrafluoroethylene (ETFE), polytetrafluoroethylene (PTFE), fluorinated ethylene propylene (FEP), polyurethane, polyimides (coats or non-coated), polyvinyl chloride (PVC), perfluoroalkoxy alkane (PFA), ethylene-vinyl acetate (EVA), polyvinylidene fluoride or polyvinylidene difluoride (PVDF), THV (a polymer of tetrafluoroethylene, hexafluoropropylene and vinylidene fluoride), PFE (Poly (fluoreny lene ethynylene)), nylon, and/or composite of nylon. In some embodiments, any of the packaging using the materials above can be co-extruded and/or laminated. In some embodiments, any of the multi-chamber packaging using the materials above can further include aluminum foil laminate, aluminum oxide laminate, or laminated or co-extruded with aluminum oxide. In some embodiments, any of the multi-chamber packaging can be laminated with a layer of alder or any other suitable adhesive. The materials from which the first layer and the second layer are selected to ensure that the two layers can be joined to hermetically seal the storage volume within which the material (or any other stored product described herein) is stored while also retaining the desired flexibility. The two layers can be joined together at one end portion and along the side edges by any suitable methods and/or mechanism, such as, for example, by heat bonding or by an adhesive.

While some embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

Although some embodiments have been described as having particular features and/or combinations of components, other embodiments are possible having a combination of any features and/or components from any of embodiments as discussed above. Aspects have been described in the general context of medical devices, and more specifically tissue packaging devices, but inventive aspects are not necessarily limited to use in medical devices and tissue packaging.

1. A flexible container, comprising:
a first layer and a second layer, at least one of the first layer or the second layer constructed from a first material, the first layer and second layer coupled together to form the flexible container having a first end portion, a second end portion, a first edge between the first end portion and the second end portion, and a second edge between the first end portion and the second end portion, the first edge opposite the second edge, the first layer and the second layer coupled together along the first edge, the second edge, and the second end portion to define a storage volume between the first layer and the second layer with an opening into the storage volume defined at the first end portion;
a primary closure including a fold region having a second material located along the opening, the fold region configured to fold over the opening such that the second material contacts the first material of one of the first layer or the second layer to seal the opening; and
a secondary closure including a tab extending past at least one of the first edge or the second edge and spaced apart from the fold region, the tab configured to secure the fold region after the fold region is folded over the opening.

2. The flexible container of claim 1, wherein the fold region is formed from a portion of the first layer that extends beyond the opening.

3. The flexible container of claim 1, wherein:
the fold region includes a fold region width; and
the secondary closure is spaced away from the fold region by a multiple of the fold region width.

4. The flexible container of claim 1, wherein the fold region extends across a full width of the first end portion.

5. The flexible container of claim 1, wherein the fold region includes a flap that extends past the first edge, the flap configured to be folded onto one of the first layer or the second layer after the fold region is folded over the opening.

6. The flexible container of claim 1, wherein the fold region includes a perforated line defining an edge of the fold region, the fold region being configured to fold about the edge and over the opening.

7. The flexible container of claim 1, wherein the first material is an extruded polymer tube.

8. The flexible container of claim 1, wherein:
the second material is an adhesive; and
the fold region includes a removable adhesive liner that covers the adhesive before use.

9. The flexible container of claim 1, wherein the second material is more rigid than the first material allowing the second material to guide the folding of the fold region over the opening.

10. The flexible container of claim 5, wherein the tab extends past the first edge and defines a connection separation region.

11. The flexible container of claim 1, wherein the secondary closure includes an adhesive or a cohesive formulated to couple the tab to the fold region after the fold region is folded over the opening.

12. The flexible container of claim 1, wherein the first layer and the second layer define a frangible region configured for opening the flexible container after the opening is sealed by at least one of the primary closure or the secondary closure.

13. The flexible container of claim 12, wherein:
the opening is a first opening; and
the frangible region includes a stress concentration riser configured to form at least one of a separation between the first layer and the second layer or a second opening through at least one of the first layer or the second layer.

14. A method of enclosing a material in a flexible container having a first end portion and a second end portion, the flexible container constructed from a first layer and a second layer coupled together to define a storage volume between the first layer and the second layer with an opening into the storage volume defined at the first end portion, the flexible container having a primary closure and a secondary closure;
separating the first layer and the second layer to expand the opening;
inserting the material into the storage volume via the expanded opening;
compressing the flexible container to expel air from the storage volume by applying a pressure against an outer surface of the flexible container progressively from the second end portion towards the opening;
removing a first adhesive liner from a fold region of the primary closure to expose a first adhesive;
folding the fold region along a perforation line to cover the opening;
sealing the fold region to the second layer via the first adhesive;
folding, after the sealing, the fold region along a length of the flexible container until the fold region overlaps the secondary closure;
removing a second adhesive liner from the secondary closure to expose a second adhesive; and
adhering the fold region to the secondary closure via the second adhesive.

15. The method of claim 14, wherein folding the fold region along the perforation line comprises folding the fold region from 2 to 6 times.

16. The method of claim 14, wherein folding the fold region along the perforation line comprises folding the fold region 4 times.

17. The method of claim 14, wherein the secondary closure includes a tab extending wider than a width of the flexible container.

18. The method of claim 17, further comprising:
wrapping the tab of the secondary closure around the fold region when adhering the fold region to the secondary closure after folding the fold region across the secondary closure such that a portion of the secondary closure adheres to one side of the fold region and the tab adheres to a second side of the fold region.

19. A flexible container system, comprising:
a first layer and a second layer, at least one of the first layer or the second layer constructed from a first material, the first layer and second layer coupled together to form the flexible container having a first end portion, a second end portion, a first edge between the first end portion and the second end portion, and a second edge between the first end portion and the second end portion, the first edge opposite the second edge, the first layer and the second layer coupled together along the first edge, the second edge, and the second end portion to define a storage volume between the first layer and the second layer with an opening into the storage volume defined at the first end portion, the first layer extending past the second layer at the opening;
a primary closure including a fold region, the fold region located along the opening and defined by a portion of the first layer and extending past the second layer, the fold region configured to form a first fold over the opening to seal the opening; and
a secondary closure spaced apart from the fold region, the secondary closure configured to form a secondary seal between the fold region and the second layer when the fold region is folded across the secondary closure.

20-21. (canceled)

22. The method of claim 14, further comprising:
coupling, before the inserting, at least one of the primary closure or the secondary closure to an attachment mechanism of a support stand so that the flexible container is maintained in a position with the first end portion being above the second end portion and the opening being expanded.
